(19) United States
(12) Patent Application Publication
(10) Pub. No.: US 2006/0182740 A1
(43) Pub. Date: Aug. 17, 2006

(54) Buffered formulations for concentrating antibodies and methods of use thereof

(75) Inventors: Tzung-Horng Yang, San Diego, CA (US); Michael J Bacica, San Diego, CA (US); Michael Labarre, San Diego, CA (US)

Correspondence Address: PILLSBURY WINTHROP SHAW PITTMAN, LLP, P.O. BOX 10500, MCLEAN, VA, 22102, US

(73) Assignee: Biogen Idec, Inc., San Diego, CA (US)

(21) Appl. No.: 10/518,434

(22) PCT Filed: Jun. 23, 2003
(86) PCT No.: PCT/US03/19652

§ 371 Date: Feb. 24, 2006

Related U.S. Application Data
(60) Provisional application No. 60/390,191, filed on Jun. 21, 2002.

(51) Int. Cl. A61K 39/395

(52) U.S. Cl. 424/133.1

(57) ABSTRACT

The present invention provides a method for producing a concentrated antibody preparation that includes the steps of: (a) obtaining an initial antibody preparation that is an aqueous solution of antibodies and histidine or acetate buffer at a concentration in the range of from about 2 mM to about 48 mM; and (b) subjecting the antibody preparation to membrane filtration so as to remove water and buffer but not antibodies from the antibody preparation, thereby producing an antibody preparation having a higher concentration of antibodies than the initial antibody preparation. The concentrated antibody preparations produced by the method have lower viscosity and are more stable than those of other formulations. The invention further includes concentrated antibody preparations produced by the method, pharmaceutical compositions made using such preparations, and therapeutic methods in which such pharmaceutical compositions are administered to treat diseases.

### RELATED APPLICATIONS

This application is related to and claims priority from U.S. Provisional Application No. 60/390,191, filed Jun. 21, 2002, entitled “Buffered Formulations For Concentrating Antibodies,” the contents of which are incorporated herein by reference in their entirety.

### FIELD OF THE INVENTION

The present invention relates to buffered antibody preparations that can be efficiently concentration by a membrane filtration process; to a process for concentrating antibodies in which such a preparation is subjected to a membrane filtration process; to a concentrated antibody preparation produced by the process; and to methods wherein concentrated antibody preparations produced by the process are used to prepare pharmaceutical antibody formulations useful for human therapy.

### BACKGROUND

Immunoglobulin G (IgG) preparations have been purified for use in human therapy since the 1940s. At present, human therapeutic immunoglobulin products are marketed commercially as 16% (w/v) (160 mg/ml) solutions for intramuscular administration, e.g., for hepatitis A prophylaxis, and as 5% (w/v) (50 mg/ml) solutions for intravenous administration, e.g., for treatment of primary immunodeficiencies, infections, and autoimmune diseases. See column 1 of U.S. Pat. No. 6,252,055, the contents of which are incorporated herein in their entirety.

Efforts to develop therapeutic monoclonal antibodies (MAbs) targeted against disease-causing antigens attained success in the late 1990s. In 1997, the FDA approved RITUXAN® (also referred to as rituximab), a chimeric anti-CD20 antibody from IDEC Pharmaceuticals Corp. and Genentech, Inc., for the treatment of non-Hodgkin's lymphoma. This was the first MAb to be approved by the FDA. Other therapeutic antibodies have since been approved by the FDA for various indications, such as Herceptin (Genentech, Inc.) for the treatment of breast cancer, Synagis (Medimmune, Inc.) for treating Respiratory Syncytial Virus infections in children, and Remicade (Centocor, Inc.) for treating Crohn's disease. (See H. Iyer et al., BioPharm, January, 2002, page 14).

Many therapeutic MAbs are currently undergoing clinical testing for FDA approval. One example is IDEC-114, an anti-CD80 MAb for treating autoimmune diseases and preventing organ transplant rejection that is described in U.S. Pat. No. 6,113,898, the contents of which are incorporated herein in their entirety. Another is IDEC-131, an anti-gp39 MAb that is also useful for treating autoimmune diseases, as described in U.S. Pat. No. 6,001,358, the contents of which are incorporated herein in their entirety. A third example is IDEC-151, an anti-CD4 MAb that is useful for T cell depletion therapy, e.g., to provide immunosuppression, as described in U.S. Pat. No. 6,136,310, the contents of which are incorporated herein in their entirety. Another therapeutic MAb being evaluated for therapeutic use is IDEC-152, an anti-CD23 antibody that inhibits IL-4-induced IgE production by B cells and is useful for treating IgE-mediated pathologies such as atopic dermatitis, allergic rhinitis, and asthma, as described in U.S. Pat. No. 6,011,138, the contents of which are incorporated herein in their entirety.

Effective treatment with therapeutic MAbs typically requires repeated administration of doses of a therapeutic preparation of MAbs that are concentrated to 100 mg/ml or greater. Therapeutic MAbs are commonly administered parenterally, by intravenous, intramuscular, or intraperitoneal delivery. The patient is frequently hospitalized during administration, because of the large volume of MAb solution that must be administered, and to permit observation of the patient's response to treatment. There is considerable interest in developing efficient methods for preparing highly concentrated preparations of therapeutic MAbs, in order to reduce the volume of solution that contains the required dosage, and so reduce the infusion time required for administration.

There is also considerable interest in developing efficient methods for preparing highly concentrated preparations of therapeutic MAbs that are suitable for subcutaneous administration, which have the advantage that they can be self-administered. Since the volume of a dose that can be administered by the subcutaneous route is relatively small (about 1 ml), the concentration of MAbs in a preparation of therapeutic MAbs that is to be administered effectively by the subcutaneous route should be in the range of 100 to 200 mg/ml. In general, it is desirable that the concentration of MAbs in a preparation of therapeutic MAbs be between 100 and 300 mg/ml (see column 4 of U.S. Pat. No. 6,252,055).

A highly concentrated solution of MAbs can be prepared by lyophilizing the antibodies, and then dissolving them in water to the desired concentration. See U.S. Pat. No. 5,608,038, the contents of which are incorporated herein in their entirety. Alternatively, a highly concentrated solution of MAbs can be produced by ultrafiltration, a technique in which a solution of MAbs is concentrated by filtering the antibody solution under pressure through a membrane filter with pores that retain the MAbs while allowing the solvent and small solute molecules to pass through. Commonly used methods for ultrafiltration are discussed below.

In preparing highly concentrated antibody preparations for pharmaceutical formulation, it is desirable to reduce the viscosity of the antibody preparation, in order to increase the rate of filtration, maximize recovery (by reducing the sticking of material to tubing, plasticware, etc.), and improve the ease of handling and the accuracy of concentration determinations. A pharmaceutical formulation with reduced viscosity is also desirable, because it can be administered more quickly to people with narrow veins, such as children. See U.S. Pat. No. 5,608,038 (column 2). It is also desirable to inhibit the formation of antibody aggregates during the preparation and concentration of therapeutic antibodies, and to remove any aggregated antibodies that have formed in solutions that are used to prepare pharmaceutical formulations, because antibody aggregates reduce the yield of biologically active antibodies, and may cause a number of adverse side-effects if they are present in a pharmaceutical formulation that is administered to a patient.

In order to inhibit aggregation and loss of biological activity when producing a highly concentrated solution of MAbs by ultrafiltration, a stabilizing additive such as a polyol, and/or a viscosity-reducing agent such as a salt or surfactant, is typically added to the composition containing the antibodies (see U.S. Pat. No. 6,171,586, and U.S. Patent Application No. 2002/0045571, the contents of both of which are incorporated herein by reference. For example, U.S. Patent Application No. 2002/0045571 describes adding a salt and/or buffer in an amount of at least 50 mM to lower the viscosity of the antibody solution during filtration. U.S. Pat. No. 5,608,038, the contents of which are incorporated herein by reference in their entirety, describes adding a saccharide such as glucose or sucrose in the antibody preparation at a concentration in the range of from 30 to 50 mg/ml in order to give the desired osmolarity and to stabilize the antibodies (see col. 2). Glycine and/or maltose are also used to stabilize antibodies in a highly concentrated antibody solution (see U.S. Pat. No. 6,252,055, the contents of which are incorporated herein by reference in their entirety).

Aggregates are efficiently removed from a concentrated antibody solution by microfiltration, a procedure which also sterilizes the antibody solution. The highly concentrated antibody preparation that is obtained by such methods can then by formulated into the pharmaceutical preparation suitable for administration to a patient.

Notwithstanding what has been previously described, there still exists a need in the art for improved methods for preparing highly concentrated antibody preparations that have lowered viscosity and reduced aggregation and are relatively free of additives, that are suitable for use in pharmaceutical formulations.

### SUMMARY AND OBJECTS OF THE INVENTION

The present invention relates to a buffered antibody preparation that is particularly suitable for being subjected to a membrane filtration process for further concentration of the antibodies; to a process for concentrating antibodies comprising subjecting such a preparation to membrane filtration; to a concentrated antibody preparation obtained by such a membrane filtration process; and to using concentrated antibody preparations obtained by the process in preparing pharmaceutical antibody formulations useful for therapy.

It is an object of the invention to provide a composition of antibodies that consists essentially of an aqueous solution of antibodies and histidine or acetate buffer at a concentration in the range of from about 2 mM to about 48 mM. The present invention also provides a composition of antibodies that consists essentially of an aqueous solution of antibodies and histidine or acetate buffer at a concentration in the range of from about 3 mM to about 48 mM, or in the range of from about 4 mM to about 45 mM, or in the range of from about 5 mM to about 40 mM. The invention further provides a composition of antibodies that consists essentially of an aqueous solution of antibodies and histidine or acetate buffer at a concentration that is in the range of from 20 mM to 25 mM. The composition of antibodies provided by the present invention can be one that is suitable for subjecting to further concentration by membrane filtration. The composition of antibodies provided by the present invention can also be one that contains a preparation of antibodies that has been concentrated by membrane filtration. Both types of compositions provided by the present invention consist essentially of an aqueous solution of antibodies and histidine or acetate buffer at a concentration in the same concentration ranges stated above.

Another object of the invention is to provide the above-described composition of antibodies that consists essentially of an aqueous solution of antibodies and histidine or acetate buffer at a concentration in the range of from about 2 mM to about 48 mM, which composition has pH in the range of from about 4.0 to about 7.5. As used in the present application, the term “about” with respect to pH means the indicated pH±0.2 pH units. For example, the composition of antibodies provided by the present invention can have pH in the range of from 4.5 to 7.0, or in the range of from 5.0 to 6.5, or in the range of from 5.5 to 6.0.

It is also an object of the invention to provide the above-described composition of antibodies that consists essentially of an aqueous solution of antibodies and histidine or acetate buffer at a concentration in the range of from about 2 mM to about 48 mM, wherein the antibodies are monoclonal antibodies. The composition of antibodies of the present invention can contain chimeric monoclonal antibodies comprising variable regions of a non-human species and human constant regions, such as PRIMATIZED® antibodies that comprise variable regions of an Old World monkey and human constant regions. The composition of antibodies of the present invention can also contain humanized monoclonal antibodies comprising hypervariable regions of a non-human species and human constant regions.

An additional object of the invention is to provide the above-described composition of antibodies that consists essentially of an aqueous solution of antibodies and histidine or acetate buffer at a concentration in the range of from about 2 mM to about 48 mM, in which the antibodies are of one or more of the isotypes selected from IgG, IgM, IgA, IgD, and IgE. For example, the composition can contain antibodies that are IgG antibodies, such as IgG₁ or IgG₄ antibodies.

Another object of the invention is to provide the above-described antibody composition that consists essentially of an aqueous solution of antibodies and histidine or acetate buffer at a concentration in the range of from about 2 mM to about 48 mM, in which the concentration of the antibodies is at least 50 mg/ml, or is at least 100 mg/ml.

A further object of the invention is to provide the above-described antibody composition that consists essentially of an aqueous solution of antibodies and histidine or acetate buffer at a concentration in the range of from about 2 mM to about 48 mM that comprises monoclonal antibodies selected from the group consisting of anti-CD80, anti-gp39, anti-CD4, anti-CD23, and anti-CD20 antibodies.

An additional object of the invention is to provide the above-described composition of antibodies that consists essentially of an aqueous solution of antibodies and histidine or acetate buffer at a concentration in the range of from about 2 mM to about 48 mM, wherein the antibodies comprise at least one monoclonal antibody selected from the group consisting the anti-CD80 antibody IDEC-114, the anti-gp39 antibody IDEC-131, the anti-CD4 antibody IDEC 151, the anti-CD23 antibody IDEC-152, and the anti-CD20 antibody RITUXAN® (rituximab).

It is another object of the invention to provide a method for producing a concentrated antibody preparation comprising the steps of (a) providing an initial antibody preparation consisting essentially an aqueous solution of antibodies and histidine or acetate buffer at a concentration in the range of from about 2 mM to about 48 mM; and (b) subjecting the initial antibody preparation to membrane filtration that removes water and buffer but not antibodies from the antibody preparation, thereby producing an antibody preparation having a higher concentration of antibodies than the initial antibody preparation.

It is an additional object of the invention to provide an improved method for producing a concentrated antibody preparation comprising the steps of (a) providing an initial antibody preparation consisting essentially of an aqueous solution of antibodies and buffer; and (b) subjecting the initial antibody preparation to membrane filtration that removes water and buffer but not the antibodies from the antibody preparation, thereby producing an antibody preparation having a higher concentration of antibodies than the initial antibody preparation; the improvement consisting of using buffer selected from histidine or acetate at a concentration in the range of from about 2 mM to about 48 mM.

A preferred method for concentrating antibodies by membrane filtration according to the present invention is ultrafiltration by tangential flow filtration. Various methods have been developed for concentrating antibodies in an antibody preparation by subjecting it to a process of membrane filtration that removes solvent and small molecules water but not antibodies from the antibody preparation. Such methods are carried out using both normal flow filtration and tangential flow filtration. The present invention provides an improvement over previously described methods for concentrating a buffered solution of antibodies by membrane filtration, the improvement being that the antibody preparation that is subjected to membrane filtration is one that consists essentially of an aqueous solution of antibodies and histidine or acetate buffer at a concentration in the range of from about 2 mM to about 48 mM.

It is another object of the present invention to provide a method for producing a pharmaceutical composition comprising antibodies as the active ingredient, comprising the steps of (a) providing an initial antibody preparation consisting essentially of an aqueous solution of antibodies and histidine or acetate buffer at a concentration in the range of from about 2 mM to about 48 mM; and (b) subjecting the initial antibody preparation to membrane filtration that removes water and buffer but not antibodies from the antibody preparation, thereby producing an antibody preparation having a higher concentration of antibodies than the initial antibody preparation; and (c) combining antibodies of the concentrated antibody preparation of step b) with one or more pharmaceutically acceptable carriers to produce a pharmaceutical composition.

It is also an object of the present invention to provide an improved method of therapy that includes the administration of a pharmaceutical composition comprising an antibody, the improvement comprising administering a pharmaceutical composition that is made by combining (a) an antibody preparation consisting essentially of an aqueous solution containing at least one therapeutically effective dose of an antibody and histidine or acetate buffer at a concentration in the range of from about 2 mM to about 48 mM that has been concentrated by membrane filtration, and (b) one or more pharmaceutically acceptable carriers to produce a pharmaceutical composition.

An additional object of the present invention is to provide a kit useful for the treatment of a mammal suffering from or predisposed to a disorder comprising at least one container containing a pharmaceutical composition that is the product of combining (a) an antibody preparation consisting essentially of an aqueous solution containing at least one therapeutically effective dose of an antibody and histidine or acetate buffer at a concentration in the range of from about 2 mM to about 48 mM that has been concentrated by membrane filtration, and (b) one or more pharmaceutically acceptable carriers; and further comprises a label or an insert indicating that said pharmaceutical composition may be used to treat said disorder.

As to each of the foregoing methods and the kit of the present invention, the concentrated antibody preparation consists essentially of an aqueous solution of antibodies and histidine or acetate buffer at a concentration in the range of from about 2 mM to about 48 mM, e.g., in the range of from about 3 mM to about 48 mM, or in the range of from about 4 mM to about 45 mM, in the range of from about 5 mM to about 40 mM, or in the range of from 20 mM to 25 mM. The same can be true for the composition of antibodies that is subjected to further concentration by membrane filtration. Either antibody preparation can also consist essentially of an aqueous solution of antibodies and histidine or acetate buffer at a concentration in the range of from about 2 mM to about 48 mM, which composition has pH in the range of from about 4.0 to about 7.5. For example, either composition of antibodies can have pH in the range of from 4.5 to 7.0, or in the range of from 5.0 to 6.5, or in the range of from 5.5 to 6.0. The antibodies each of the foregoing methods and kit can be chimeric monoclonal antibodies comprising variable regions of a non-human species and human constant regions, such as PRIMATIZED® antibodies that comprise variable regions of an Old World monkey and human constant regions. The antibody compositions can also contain humanized monoclonal antibodies comprising hypervariable regions of a non-human species and human constant regions. In addition, the antibodies each of the foregoing methods and kit can be one or more of the isotypes selected from IgG, IgM, IgA, IgD, and IgE. For example, they can be IgG antibodies such as IgG₁ or IgG₄ antibodies. The concentration of the antibodies in the concentrated antibody preparations of each of the foregoing methods and kit can be at least 50 mg/ml, or at least 100 mg/ml. The antibody compositions can contain monoclonal antibodies selected from the group consisting of anti-CD80, anti-gp39, anti-CD4, anti-CD23, and anti-CD20 antibodies. For example, the antibody compositions can comprise at least one monoclonal antibody selected from the group consisting the anti-CD80 antibody IDEC-114, the anti-gp39 antibody IDEC-131, the anti-CD4 antibody IDEC 151, the anti-CD23 antibody IDEC-152, and the anti-CD20 antibody RITUXAN® (rituximab). Antibody compositions of the foregoing methods and kit can be used in an improved method of therapy that comprises administering a therapeutically effective dose of therapeutic antibody to a patient suffering from a disease selected from the group consisting of cancer, allergic disorders, autoimmune diseases, and lymphoma

### BRIEF DESCRIPTION OF THE FIGURES

FIG. 1 schematically depicts direct flow filtration (DFF). The feed, i.e., the solution to be filtered, is forced directly toward the membrane as shown in FIG. 1. The smaller molecules pass through the pores as the filtrate while the larger antibodies are retained by the membrane. The molecules larger than the pores are shown aggregating at the membrane surface and forming a gel.

FIG. 2 is a graph showing that the flux rate during DFF decreases rapidly as filtration proceeds, because the antibodies aggregate at the membrane surface and form a gel that blocks the flow of the smaller molecules through the pores.

FIG. 3 schematically depicts tangential flow filtration (TFF). Once the feed is introduced into the system, the solution circulates so that the sample flows across the surface of the membrane while pressure in the solution forces smaller molecules in the solution through the pores of the membrane as filtrate. The solution and antibody molecules that remain between the membranes form the retentate.

FIG. 4 is a graph showing that the flux rate during TFF decreases gradually as filtration proceeds.

FIG. 5 is a graph that shows the dependence of filtration flow rate on antibody concentration for solutions containing three different buffers at pH 5.5 and pH 6.0. From the data plotted in the graph, it can be seen that filtration flow rate at a wide range of antibody concentrations is markedly greater with histidine and acetate buffers than with citrate buffer. There do not appear to be significant differences between flow rates achieved at pH 5.5 and pH 6.0.

FIG. 6 is a graph that shows the change in OD320, a measure of turbidity, with increases in antibody concentration over the course of TFF, for solutions containing three different buffers at pH 5.5 and pH 6.0. It can be seen from the graph that the formulation containing citrate buffer had the highest turbidity, there was intermediate turbidity in the acetate-containing formulation, and the formulation containing histidine had the lowest turbidity.

FIG. 7 is a bar graph representing the kinematic viscosities of solutions of IDEC-114 formulated at 135 mg/ml with different buffers at pH 5.5 and 6.0. The citrate-containing formulations had significantly higher viscosities than the others. Viscosities of formulations at pH 6.0 also are consistently higher than those at pH 5.5.

### DETAILED DESCRIPTION OF THE INVENTION

Antibody therapeutics can be used successfully to treat a number of oncology- and immune system-related indications; however, large dosages of an antibody drug are often required if the drug is to be therapeutically effective. In order to deliver a therapeutically effective dosage of an antibody to a patient by intravenous or subcutaneous routes, the concentration of the antibody preparation usually must be high, a requirement that frequently creates difficulties, both in preparing the drug and in maintaining it in stable form.

The present invention is directed to providing compositions and methods that permit the production of highly concentrated, stable antibody preparations of relatively low viscosity that are substantially free of aggregates and are suitable for use in a pharmaceutical formulation.

In one embodiment, the present invention provides a method for producing a concentrated antibody preparation. The steps of the method comprise:

(a) providing an initial antibody preparation consisting essentially of an aqueous solution of antibodies and histidine or acetate buffer at a concentration in the range of from about 2 mM to about 48 mM; and

(b) subjecting the initial antibody preparation to membrane filtration that removes water and buffer but not the antibodies from the antibody preparation, thereby producing an antibody preparation having a higher concentration of antibodies than the initial antibody preparation.

As used in the present application, the term “about” with respect to concentrations of histidine or acetate means the indicated concentration ±3% of the indicated concentration. Antibody compositions of the invention consist essentially of an aqueous solution of antibodies and histidine or acetate buffer at any concentration in the range of from about 2 mM to about 48 mM. For example, the concentration of histidine or acetate buffer can be in the range of from about 3 mM to about 48 mM, or in the range of from about 4 mM to about 45 mM, or in the range of from about 5 mM to about 40 mM. The concentration of histidine or acetate buffer in the antibody composition can also be in the range of from 20 mM to 25 mM.

Antibody compositions of the invention include initial antibody preparations that are suitable for subjecting to further concentration by membrane filtration, and they also include any antibody preparations that have been concentrated by membrane filtration. Whether they are initial antibody preparations or antibody preparations that have been concentrated by membrane filtration, the antibody compositions provided by the present invention consist essentially of an aqueous solution of antibodies and histidine or acetate buffer at a concentration in the range of from about 2 mM to about 48 mM.

The present invention provides and includes compositions of concentrated antibodies that are prepared by practicing the foregoing method, as well as pharmaceutical formulations comprising the concentrated antibody preparations that are made using concentrated antibodies produced by the method of the invention.

The invention springs from the unexpected observation that low concentrations of acetate or histidine buffer (of from about 2 mM to about 48 mM) are able to stabilize an antibody preparation during concentration by membrane filtration, lowering the viscosity of the antibody solution, and suppressing aggregation, to an extent that equals or surpasses the stabilizing effects that have been achieved using other, more complex formulations described in the art. The invention provides a method whereby ultrafiltration is used to produce a highly concentrated, stable antibody preparation that contains a relatively low level of aggregates. The resulting concentrated antibody preparation consists essentially of an aqueous solution of antibodies and histidine or acetate buffer at a concentration in the range of from about 2 mM to about 48 mM, and is free of additives such as polyols, saccharides, glycerin, salts, and high buffer concentrations (over 50 mM) that are presently used in the art to stabilize and reduce viscosity of concentrated antibody preparations.

As used herein, a “stable” antibody preparation is one in which the protein therein essentially retains its physical stability and/or chemical stability and/or biological activity upon storage. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10: 29-90 (1993), for example. Stability can be measured at a selected temperature for a selected time period. A reasonably stable antibody preparation is one that is stable at room temperature (about 30° C.) or at 40° C. for at least 1 month, and/or is stable at about 2-8° C. for at least 1 year, and following freezing (e.g., to −70° C.) and thawing of the formulation. A protein “retains its physical stability” in a pharmaceutical formulation if it shows no signs of aggregation, precipitation and/or denaturation upon visual examination of color and/or clarity, or as measured by UV light scattering or by size exclusion chromatography. A protein “retains its chemical stability” in a pharmaceutical formulation, if the chemical stability at a given time is such that the protein is considered to still retain its biological activity as defined below. Chemical stability can be assessed by detecting and quantifying chemically altered forms of the protein. Chemical alteration may involve size modification (e.g. clipping) which can be evaluated using size exclusion chromatography, SDS-PAGE and/or matrix-assisted laser desorption ionization/time-of-flight mass spectrometry (MALDI/TOF MS), for example. Other types of chemical alteration include charge alteration (e.g. occurring as a result of deamidation) which can be evaluated by ion-exchange chromatography, for example. An antibody “retains its biological activity” in a pharmaceutical formulation, if it has a significant amount (e.g., about 90%) of the biological activity of the antibody that was exhibited at the time the pharmaceutical formulation was prepared. For example, biological activity can be determined in an antigen binding assay. See U.S. Pat. No. 6,171,586. The types of “biological activity” assays that are relevant for any particular antibody generally depend on the biological role(s) of the specific molecule targeted by the antibody, and the biological consequences of the binding of the antibody to that target. Persons skilled in the art are generally familiar with many such assays.

The antibody preparations consisting essentially of an aqueous solution of antibodies and histidine or acetate buffer at a concentration in the range of from about 3 mM to about 48 mM, that are produced by the method of the present invention generally have pH in the range of from about 4.0 to about 7.5. For example, the antibody preparations can have pH in the range of from 4.5 to 7.0, or in the range of from 5.0 to 6.5; or in the range of from 5.5 to 6.0. Such solutions can be made by common methods well-known to those in the art. Preferably, the acetate buffer is Na-acetate, and the histidine buffer is histidine HCl; however, the invention can also be practiced successfully by employing any available buffers in which histidine or acetate are conjugated with counterions/acid-base components other than Na⁺ and Cl⁻ when adjusting the pH to the above-stated values.

The antibodies of the present invention may be of any isotype. For example, they may be of any of the major isotype classes, IgM, IgG, IgA, IgE and IgD. Antibodies of the various subclasses of each isotype are effectively concentrated by the present invention. For example, highly concentrated preparations of active, non-aggregated antibodies of sub-classes IgG₁, IgG₂, IgG₃ and IgG₄ of the IgG isotype can be produced by the present invention. Preparations of antibodies that can be concentrated successfully using the present invention can contain a single type of antibody, or they can contain two or more different types of antibodies.

The term “antibody” as used herein is intended to include antibody fragments having a specific binding activity of interest. The present invention can be used for concentrating such fragments of any antibody isotype, including antibody fragments such as Fab, F(ab′)₂, Fv, as well as Fc, or pFc′ fragments. Antibodies can be fragmented and the fragments screened to identify those having a specific binding activity of interest using conventional techniques known in the art. For example, F(ab′)₂ fragments are generated by treating antibody with pepsin, and reduction of the disulfide bridges of F(ab′)₂ fragments produces Fab fragments

Highly concentrated preparations of active, non-aggregated bispecific or multispecific antibodies can also be produced using the present invention. Bispecific and multispecific antibodies have binding specificities for at least two different epitopes, where the epitopes are usually from different antigens. While such molecules normally will only bind two different epitopes (i.e. bispecific antibodies), the invention can also be practiced with antibodies with additional specificities such as trispecific antibodies. Examples of therapeutic multispecific antibodies suitable for use with the present invention are described, for example, in U.S. Pat. No. 6,171,586.

The present invention effectively produces concentrated preparations of active and non-aggregated monoclonal antibodies having antibody concentrations in the range of from 25 to 350 mg/ml. For example, concentrated preparations of monoclonal antibodies having antibody concentrations in the range of from 50 to 150 mg/ml, e.g., having an antibody concentration of 50, 60, 70, 80, 90, 100, 110, 120, 130, 140, or 150 mg/ml, are efficiently produced by the present invention. Concentrated preparations of monoclonal antibodies having antibody concentrations in the range of from 50 to 250 mg/ml, e.g., having an antibody concentration of 50, 75, 100, 125, 150, 175, 200, 225, or 250 mg/ml, are also efficiently produced by the present invention.

The invention may also be used for producing highly concentrated preparations of recombinant antibodies, particularly chimeric antibodies and humanized antibodies, which are a special type of chimeric antibody. In general, chimeric antibodies are antibodies that have light and heavy chain variable regions of one animal species, and constant regions of a different species. For example, a chimeric antibody having little or no immunogenicity in humans can be obtained by replacing the light and heavy chain variable regions of a human antibody with those of a non-human primate, e.g., an Old World monkey. Such antibodies are referred to as “PRIMATIZED®” antibodies, which are described in U.S. Pat. No. 6,136,310, and in U.S. Pat. No. 5,658,570, the contents of which are incorporated herein in their entirety.

“Humanized” forms of non-human antibodies are chimeric antibodies that contain minimal polypeptide sequences derived from the non-human immunoglobulin. The minimal polypeptide sequences of a non-human immunoglobulin required to retain specificity for antigen are typically the hypervariable regions (i.e., the complementarity-determining regions, CDRs 1-3), and a humanized antibody can be made by replacing the residues of the three hypervariable regions of a recipient human immunoglobulin with residues from the hypervariable regions having the desired specificity, affinity, and capacity from a (donor) antibody of a non-human mammal such as mouse, rat, rabbit or nonhuman primate. In some instances, some or all of one or more of the framework residues of the human immunoglobulin are replaced by corresponding non-human residues. Humanized antibodies may also comprise residues that are not found in the recipient antibody or in the donor antibody; such modifications are usually made to further refine or optimize antibody performance. A humanized antibody can comprise substantially all of at least one, and typically two, variable domains, in which all or substantially all of the hypervariable regions correspond to those of a non-human immunoglobulin and all or substantially all of the framework regions are those of a human immunoglobulin sequence. See Jones et al., Nature 321:522-525 (1986); Riechmann et al, Nature 332:323-329 (1988); and Presta, Curr. Op. Struct. Biol. 2:593-596 (1992), the contents of which are incorporated herein by reference in their entirety.

In a useful embodiment of the present invention, the composition of monoclonal antibodies that is concentrated by membrane filtration comprises monoclonal antibodies selected from the group consisting of anti-CD80, anti-gp39, anti-CD4, anti-CD23, and anti-CD20 antibodies. Such antibodies have been described in the scientific literature and can be prepared by routine methods. For example, the composition of monoclonal antibodies that is concentrated by membrane filtration can comprises at least one therapeutically effective dose of one or more of the therapeutic monoclonal antibodies selected from the group consisting of RITIXAN®, IDEC-114, IDEC-131, IDEC-151, and IDEC-152 antibodies.

RITUXAN® (also referred to as “rituximab”), is a chimeric anti-CD20 antibody from IDEC Pharmaceuticals Corp. and Genentech, Inc., for the treatment of non-Hodgkin's lymphoma, and is described in U.S. Pat. No. 6,399,061, the contents of which are incorporated herein in their entirety.

IDEC-114 is an anti-CD80 MAb for treating autoimmune diseases and preventing organ transplant rejection that is described in U.S. Pat. No. 6,113,898. IDEC-131 is an anti-gp39 MAb that is also useful for treating autoimmune diseases, as described in U.S. Pat. No. 6,001,358.

IDEC-151 is an anti-CD4 MAb that is useful for T cell depletion therapy, as described in U.S. Pat. No. 6,136,310.

IDEC-152 is an anti-CD23 antibody that inhibits IL-4-induced IgE production by B cells and is useful for treating IgE-mediated pathologies such as atopic dermatitis, allergic rhinitis, and asthma, as described in U.S. Pat. No. 6,011,138. The contents of the U.S. patents describing making and using these therapeutic MAbs are incorporated herein by reference in their entirety.

Concentration by Membrane Ultrafiltration

The present invention stems from the discovery that the stability and viscosity of a antibody preparation subjected to concentration by membrane ultrafiltration is sensitive to the type of buffer present in the preparation, and that certain buffers, in particular, histidine and acetate, unexpectedly lower the viscosity of an antibody preparation, reduce antibody aggregation, and increase the rate of concentration of the antibody preparation by membrane filtration, relative to what is obtained using other buffers. As a result, it is found that a preparation consisting essentially of antibodies and histidine or acetate at a concentration in the range of from about 3 mM to about 48 mM can be concentrated efficiently by ultrafiltration to a high concentration with retention of biological activity and relatively little aggregation, even in the absence of a stabilizing or viscosity-reducing additive such as a surfactant, a polyol, a saccharide, a salt, of high buffer concentration (above 50 mM). The invention operates effectively when the antibodies were previously lyophilized, and also when the antibodies have never been lyophilized.

While diverse methods for preparing and purifying therapeutic MAbs have been developed, they typically have in common a final step of concentration by ultrafiltration that precedes formulation of the final product—the pharmaceutical preparation that is to be administered. Ultrafiltration of MAbs is generally carried out by filtering the antibody solution under pressure through a membrane filter with pores that retain polypeptides of 50-200 kilodaltons while allowing smaller molecules to pass through. Membrane filters with pores that retain polypeptides 30-50 kilodaltons can be used to concentrate MAbs by ultrafiltration with good result; and membranes with pores that retain polypeptides as small as 10 kilodaltons can also be used, especially if antibody fragments are being concentrated. The efficiency of the ultrafiltration operation can be affected by the viscosity of the solution, the solubility, and amount of aggregates of the protein.

Diafiltration is the fractionation process in which smaller molecules are washed through the membrane, leaving the larger molecules of interest in the retentate (the solution retained on the other side of the membrane).

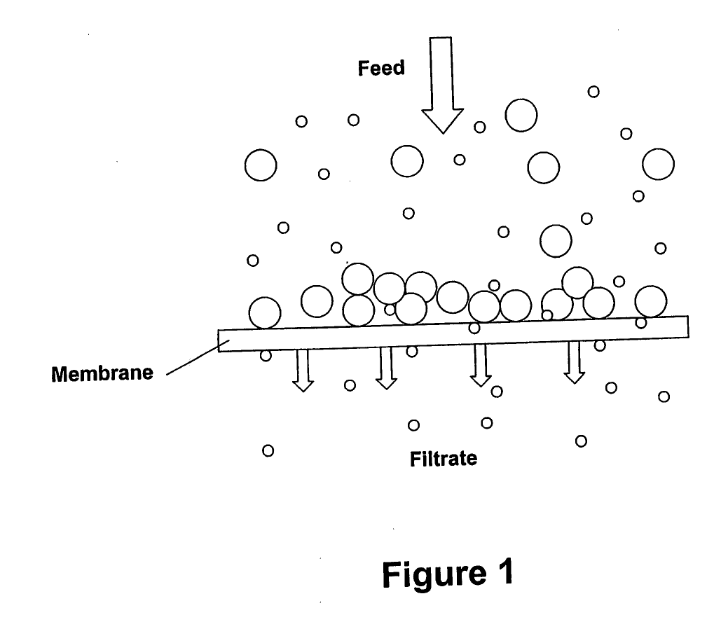

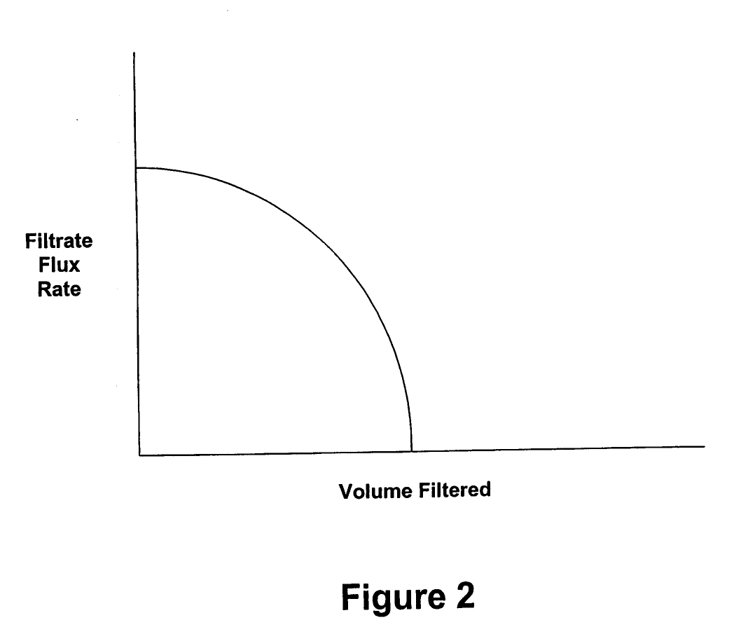

Two membrane filtration methods are commonly used for ultrafiltration. In direct flow filtration (DFF) the feed (the solution to be filtered) is forced directly toward the membrane as shown in FIG. 1. As a result, molecules larger than the pores aggregate at the membrane surface and form a gel that blocks the flow of the smaller molecules through the pores, so that the flux rate decreases rapidly as filtration proceeds, as shown in FIG. 2. DFF is also called “normal flow filtration” because the fluid flow occurs in a direction normal to the membrane surface. The protein solution is often stirred during DFF in order to keep the retained protein from aggregating and blocking the pores of the membrane. Surprisingly, depending on the conditions (e.g., pressure and flow rates), the shear forces caused by circulating retentate through a TFF system (described below) may cause more aggregation and precipitation that is caused by stirring a protein solution during DFF (see U.S. Pat. No. 6,252,055, Example 3, columns 10-11).

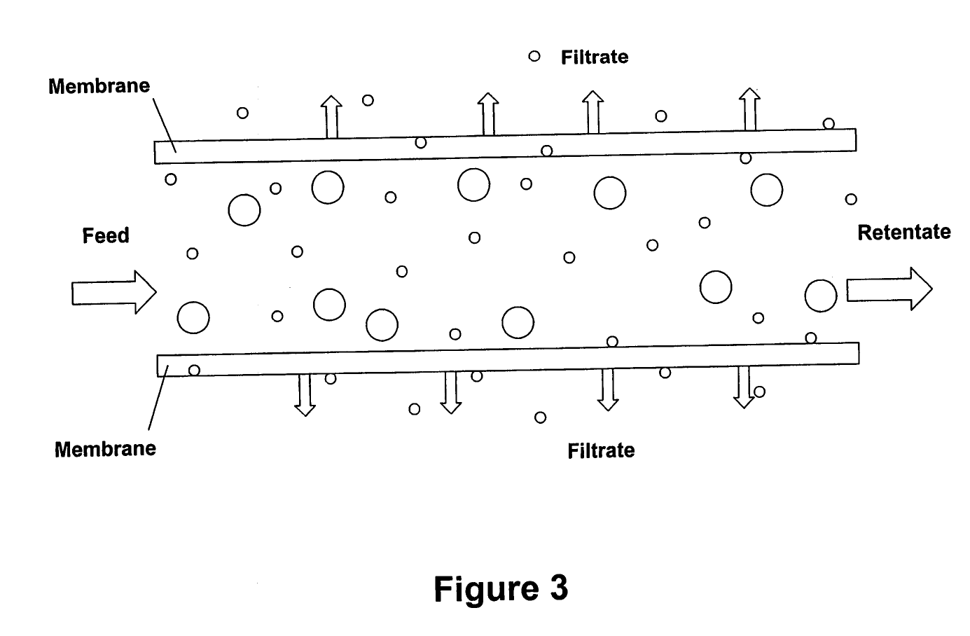

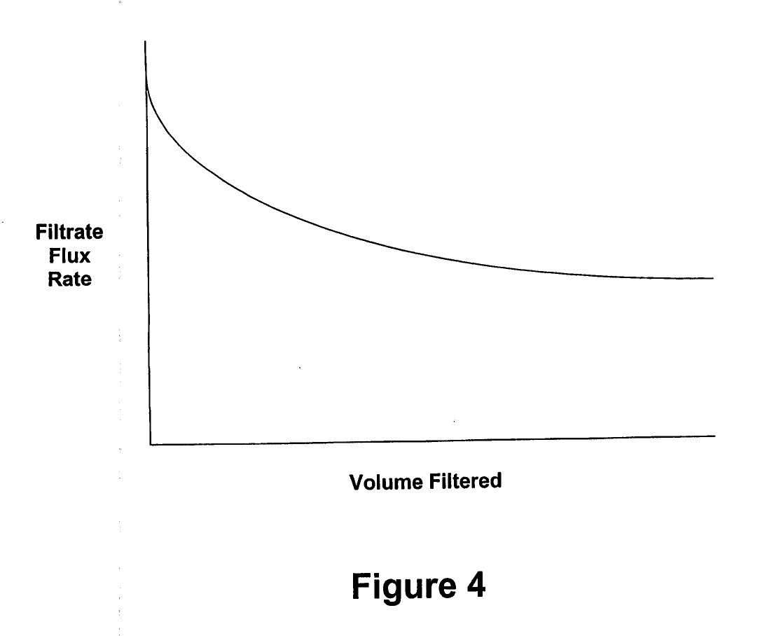

The other main ultrafiltration process is tangential flow filtration (TFF), in which the sample flows across the surface of the membrane as pressure on the solution forces smaller molecules in the solution outwards through the pores of the membrane, as shown in FIG. 3. The flow of solution across the membrane during TFF helps prevent a gel of aggregated molecules from forming on the surface of the membrane of that blocks the pores and prevents smaller molecules from passing through. As a result, the flux rate for TFF drops off much more slowly as filtration proceeds than occurs during DFF, as shown in FIG. 4.

The present invention is operative with any membrane ultrafiltration method for preparing highly concentrated solutions of antibodies. For example, the present invention operates efficiently in conjunction with the use of TFF for preparing highly concentrated solutions of MAbs that are useful in formulating pharmaceutical MAb preparations. TFF systems for performing ultrafiltration of MAB solutions are commercially available, for example, from Millipore Corp. (Bedford, Mass.), Pall Corp. (East Hills, N.Y.), or Marcon Wines and Filters (Oakville, Ontario). The use of TFF to prepare a concentrated antibody solution is also described in U.S. Pat. No. 6,252,055, the contents of which are incorporated herein in their entirety.

In addition to concentrating, a TFF system can be used to exchange buffers or to reduce the concentration of undesirable species, e.g., to the lower concentration of salt, in the preparation. This is done by introducing fresh buffer while filtering under pressure to remove the original solvent and other small molecules that are not retained by the filter. By concentrating a solution to half its volume and adding new buffer four times, it is possible to remove over 96% of the salt in a preparation. More than 99% of the original buffer in a solution can be replaced by adding up to 7 volumes of new buffer during continuous diafiltration.

The present invention is well suited to being practiced using ultrafiltration by TFF. The initial antibody preparation, or “feed”, can be a composition of antibodies that consists essentially of an aqueous solution of antibodies and histidine or acetate buffer at a concentration in the range of from about 2 mM to about 48 mM. The “feed” can also contain a salt or other small molecule solute, in addition to the antibodies and histidine or acetate buffer, without interfering with the effectively operation of the invention, since such small molecule components will pass through the membrane and be removed by diafiltration. Thus, the concentrated antibody preparation that is ultimately produced will consist essentially of an aqueous solution of antibodies and histidine or acetate buffer at a concentration in the range of from about 2 mM to about 48 mM, even if the feed does not have such composition.

Pharmaceutical Formulations

The terms “pharmaceutical formulation” and “pharmaceutical composition” as used herein refer to preparations which are in such form as to permit the biological activity of the active ingredients to be unequivocally effective, and for which any toxic effects are outweighed by the therapeutic effects. “Pharmaceutically acceptable” carriers (vehicles, additives) are those which can reasonably be administered to a subject mammal to provide an effective dose of the active ingredient employed.

Concentrated antibody preparations prepared according to the present invention may be used to prepare pharmaceutical formulations by combining a concentrated antibody preparation consisting essentially of an aqueous solution of antibodies and histidine or acetate buffer at a concentration in the range of from about 2 mM to about 48 mM produced according to the disclosed invention with one or more pharmaceutically acceptable carriers to produce a pharmaceutical composition.

Such a pharmaceutical composition may optionally be prepared to include one or more additional therapeutic ingredients. The carrier(s) must be “acceptable” in the sense of being compatible with the other ingredients of the formulation and not deleterious to the recipient thereof.

The antibodies and pharmaceutical compositions of the invention are particularly useful for parenteral administration, i.e., subcutaneously, intramuscularly or intravenously. The compositions for parenteral administration will commonly comprise a solution of an antibody or fragment thereof of the invention or a cocktail thereof dissolved in an acceptable carrier, preferably an aqueous carrier. A variety of aqueous carriers may be employed, e.g., water, buffered water, 0.4% saline, 0.3% glycine, ethanol, and the like. These solutions are sterile and generally free of particulate matter. These solutions may be sterilized by conventional, well-known sterilization techniques; e.g., by microfiltration. The compositions may contain pharmaceutically acceptable auxiliary substances as required to approximate physiological conditions such as pH adjusting and buffering agents, etc. The concentration of the antibody or fragment thereof of the invention in such pharmaceutical formulation can vary widely, i.e., from less than about 0.5%, usually at or at least about 1%, to as much as 15% or 20% by weight, and will be selected primarily based on fluid volumes, viscosities, etc., according to the particular mode of administration selected.

Thus, a pharmaceutical composition of the invention for intramuscular injection could be prepared to contain 1 ml sterile buffered water, and 50 mg. of an antibody or fragment thereof of the invention. Similarly, a pharmaceutical composition of the invention for intravenous infusion could be made up to contain 250 ml. of sterile Ringer's solution, and 150 mg. of an antibody or fragment thereof of the invention.

Actual methods for preparing parenterally administrable compositions are well-known or will be apparent to those skilled in the art, and are described in more detail in, for example, Remington's Pharmaceutical Science, 15th ed., Mack Publishing Company, Easton, Pa., hereby incorporated by reference herein.

Therapeutic Uses

The present invention provides an improvement to a method of therapy that includes the administration of a pharmaceutical composition comprising an antibody. The improvement comprises administering a pharmaceutical composition that is made by combining (a) an antibody preparation consisting essentially of an aqueous solution containing at least one therapeutically effective dose of an antibody and histidine or acetate buffer at a concentration in the range of from about 2 mM to about 48 mM that has been concentrated by membrane filtration, and (b) one or more pharmaceutically acceptable carriers. As disclosed herein, the concentrated antibody preparation comprising histidine or acetate buffer according to the present invention has viscosity and stability that are suitable for use in a pharmaceutical composition, and are generally favorable relative to the viscosity and stability provided by other preparations. As described above, the pH of the concentrated antibody preparation is generally in the range of from 4.5 to 7.0.

The concentrated antibody preparation used for the improved method of therapy may comprise a therapeutically effective dose of therapeutic chimeric monoclonal antibodies, including antibodies that are PRIMATIZED® or otherwise humanized. The disclosed pharmaceutical composition comprising a concentrated antibody preparation comprising histidine or acetate buffer at a concentration in the range of from 5 mM to 40 mM is administered used in the same manner as the pharmaceutical compositions comprising a therapeutically effective dose of therapeutic antibodies of the prior art.

Treatment of disease by administering therapeutic monoclonal antibodies selected from the group consisting of RITUXAN® (rituximab), IDEC-114, IDEC-131, IDEC-151, and IDEC-152 antibodies is also beneficial.

The above-described improved method of therapy comprises, for example, administering a therapeutically effective dose of therapeutic antibody to a patient suffering from a disease selected from the group consisting of cancer, allergic disorders, and autoimmune diseases. For example, administration of monoclonal antibodies selected from the group consisting of anti-CD80, anti-gp39, anti-CD4, anti-CD23, and anti-CD20 antibodies is known to provide therapeutic benefit to a patient in need of such administration.

A useful embodiment of the invention comprises administering a pharmaceutical composition comprising a therapeutically effective dose of therapeutic antibody to a patient suffering from a disease selected from the group consisting of cancer, allergic disorders, autoimmune diseases, and lymphoma, in order to treat the disease, i.e., to provide therapeutic benefit by inhibiting or preventing the disease, or by alleviating the disease's pathological symptoms.

A “Kit” Containing a Concentrated Preparation of Therapeutic Antibodies

The present invention further provides a kit that is useful for the treatment of a mammal suffering from, or predisposed to, a disorder. “Treatment” as used herein refers both providing therapeutic benefit to a patient suffering from an ongoing disease, as well as to prophylactic or preventative measures. Inside the kit is at least one container containing a pharmaceutical composition that is the product of combining (a) an antibody preparation consisting essentially of an aqueous solution containing at least one therapeutically effective dose of an antibody and histidine or acetate buffer at a concentration in the range of from about 2 mM to about 48 mM that has been concentrated by membrane filtration, and (b) one or more pharmaceutically acceptable carriers. The kit further comprises a label or an insert indicating that said pharmaceutical composition may be used to treat the disorder. The kit may be contain a therapeutically effective dose of therapeutic monoclonal or polyclonal antibodies. In one useful embodiment, the therapeutic antibody is an IgG antibody. In another useful embodiment, the therapeutic antibody is a monoclonal antibody; for example, a primatized monoclonal antibody.

In an especially preferred embodiment, the kit contains a therapeutically effective dose of therapeutic antibody that is useful for treating a disorder selected from the group consisting of cancer, allergic disorders, autoimmune diseases, and lymphoma. In a preferred embodiment, the therapeutic antibody is selected from the group consisting of anti-CD80, anti-gp39, anti-CD4, anti-CD23, and anti-CD20 antibodies. In a particularly preferred embodiment, the therapeutic antibody is selected from the group consisting of Rituxan, IDEC-114, IDEC-131, IDEC-151, and IDEC-152 antibodies.

### EXAMPLE

Tangential flow filtration is one of the most commonly used techniques in the processing steps to concentrate protein and dialfiltrate the material for the final formulation. The success of its operation could significantly influence product yield and stability. Thus it is important to explore the factors that might improve the efficiency of this operation. To this end, in the present study, we examine the effects of buffer species and pH on the performance of tangential flow filtration and their effects on product stability. This example demonstrates that MAb preparations formulated with relatively low concentrations of acetate or histidine buffers (5-40 mM) have lower viscosity and less aggregation relative to the results obtained with a preparation of the same MAb formulated with a different other buffer (e.g. citrate).

Tangential flow filtration (TFF) is commonly used for diafiltration and concentration of a MAb preparation in the final steps of preparing an highly concentrated aqueous MAb solution suitable for use as a pharmaceutical formulation. The efficiency of TFF can be affected by the viscosity of the solution, the solubility of the protein, and extent to which the protein has formed aggregates in the solution.

Materials and Methods

Stock solutions of IDEC-114 MAbs at a concentration of 10 mg/ml in 10 mM citrate (pH 6.5) and 150 mM NaCl or in 25 mM sodium acetate (pH 6.0) and 220 mM glycine, stored aseptically at 2-8° C., were obtained. DEC-114 MAbs are primatized antibodies—chimeric, recombinant IgG1 MAbs that have human constant regions and macaque monkey variable regions that bind CD80. The stock IDEC-114 MAb solutions were concentrated to 25 mg/ml by diafiltration at room temperature, using a LabScale Tangential Flow Filtration (TFF) System equipped with Pellicon XL (PLCTK 30) membrane cassettes (Millipore Corp., Bedford, Mass.). Six aqueous solutions consisting essentially of IDEC-114 MAbs at 25 mg/ml and a selected buffer at a desired pH were then prepared by diafiltration at room temperature by exhanging one volume of antibody buffer for eight volumes of each of the following test buffers: 20 mM sodium acetate, pH 5.5 and 6.0; 20 mM sodium citrate, pH 5.5 and 6.0; and 20 mM histidine/HCl, pH 5.5 and 6.0. The chemicals used to prepare the buffer solutions were: sodium acetate (Sigma, S-1304); sodium citrate (Fisher, S279-500), and histidine (JT Baker, product # 2080.06).

The samples were then further concentrated in the Labscale TFF System until the permeate flow rate approached 1 ml/min, at which time the antibody solutions were concentrated to above 150 mg/ml. The time required to achieve a concentration of 150 mg/ml was recorded. To maintain the uniformity of all the operations, the system flow rate was fixed at 80 ml/min, under optimal retention pressure, during the whole process.

Periodically during concentration by TFF, small aliquots of the MAb solutions were withdrawn for determination of protein concentration and measurement of viscosity and turbidity, at which time the permeate flow rate was also recorded. After TFF, samples were removed from the system and passed through an Acrodisc PF Syringe Filter 0.8/0.2 μm Supor membrane (Gelman Laboratory) to remove soluble aggregates.

Effects of Buffer Species and pH on the Operational Efficiency of TFF

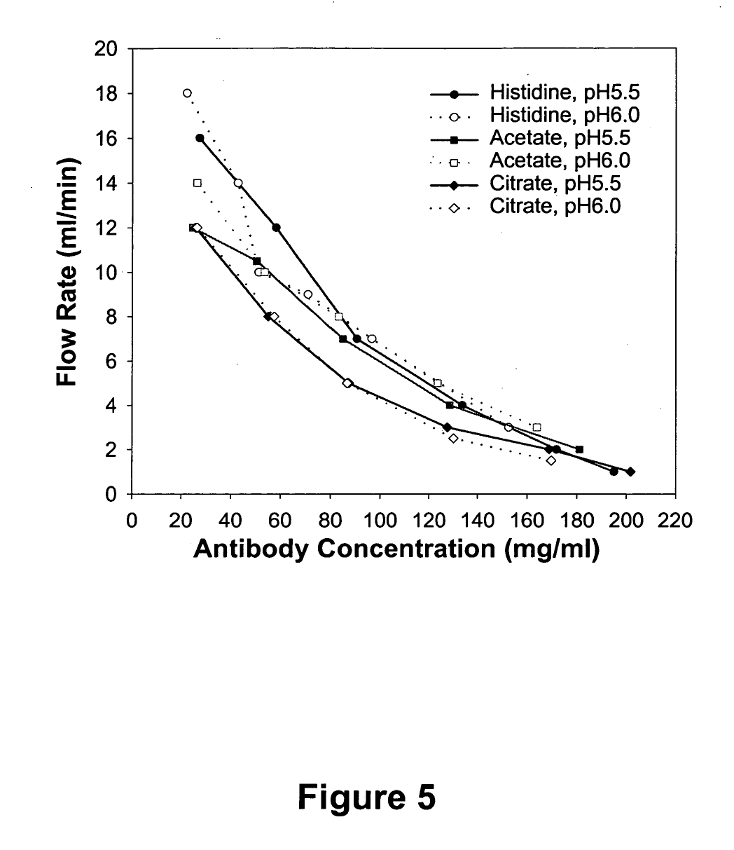

Protein concentrations were determined by UV spectrophotometric scan over the course of TFF. The samples were accurately diluted to 100× or 200× in water, depending on the concentration, and the absorbance at 280 nm was read with a Shimadzu Multispec-1501 photo diode array spectrophotometer against water as blank. FIG. 5 shows the permeate flow rate at different concentrations of antibody during the TFF process, from which it can clearly be seen that the permeate flow rates followed the trend: histidine>acetate>citrate. There was no trend regarding the pH effect on the flow rate. At concentrations above 80 mg/ml the permeate flow rate reduced, and we saw no significant difference between the flow rates of histidine- and acetate-formulated samples; however, the permeant flow rates for both of these were significantly higher than for the citrate-formulated material at the two pH values tested. The time it took to concentrate 240 ml of 25 mg/ml IDEC-114 to 150 mg/ml is listed on Table I. On average, antibody solutions formulated with citrate took about 30% more time to concentrate than those formulated with acetate, and about 50% more time than those formulated with histidine.

TABLE I

| Formulation | Time (mm) |
|---|---|
| 20 mM histidine/HC1, pH 5.5 | 19.0 |
| 20 mM histidine/HC1, pH 6.0 | 20.4 |
| 20 mM sodium acetate, pH 5.5 | 23.4 |
| 20 mM sodium acetate, pH 6.0 | 20.5 |
| 20 mM sodium citrate, pH 5.5 | 27.7 |
| 20 mM sodium citrate, pH 6.0 | 30.4 |

Turbidity

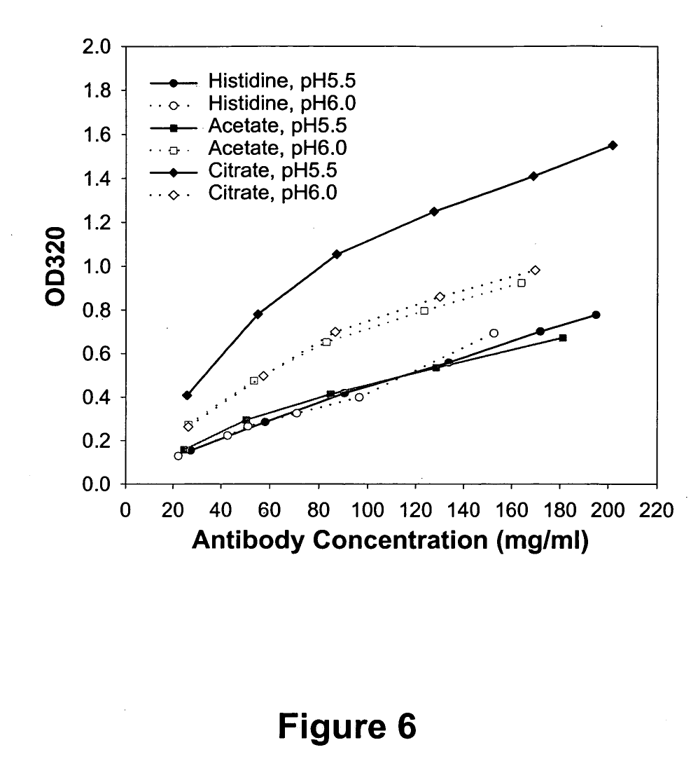

During TFF operation, the antibody molecules were continuously pumped through the system for numerous cycles, and so are subjected to strong shearing forces that could potentially result in aggregation and increase the turbidity of the solution. FIG. 6 depicts the turbidity profile of the formulated antibody, as measured by OD320 over the course of the concentration process. It is obvious that-citrate formulated MAb solution had much higher turbidity than acetate- and histidine-formulated solutions at both pH values. With the exception of the pH 6.0 acetate formulation, the latter two buffers had very similar profiles. This result indicates that histidine and acetate buffers offer significantly better protection against aggregation of antibody molecules relative to citrate buffer.

Agitation Assay

To further measure the stability of the concentrated MAbs an accelerated aggregation study was performed. 3 ml of each filtered formulation from the TFF process were put into a sterile 5 cc type I glass vial, which was stoppered with a teflon-faced, gray, butyl rubber stopper, and capped with crimp seal. The vials then were put on a shaker set at 700 rpm and agitated at room temperature for 72 hours. Each concentrated antibody formulation was filtered through a 0.2μ membrane and its concentration was adjusted to 150 mg/ml. OD320 and OD 580 readings taken before and after the agitation are shown in Table II. Although the initial OD320 readings were all relatively low and were very similar to each other, after agitation, both citrate-formulated antibodies had the highest turbidity, followed by histidine and acetate. A similar trend is reflected in the OD580 measurements. We did not find any specific pH trend for the turbidity, but within the same buffer, different pH values resulted in different levels of aggregation.

TABLE II

| | OD₃₂₀ | | OD₅₈₀ | |
|---|---|---|---|---|
| Formulation | Before | After | Before | After |
| 20 mM Histidine, pH 5.5 | 0.292 | 0.868 | 0.007 | 0.135 |
| 20 mM Histidine, ph 6.0 | 0.306 | 1.010 | 0.007 | 0.168 |
| 20 mM Acetate, pH 5.5 | 0.291 | 0.868 | 0.011 | 0.148 |
| 20 mM Acetate, pH 6.0 | 0.282 | 0.610 | 0.012 | 0.082 |
| 20 mM Citrate, pH 5.5 | 0.375 | >2.0 | 0.017 | 0.470 |
| 20 mM Citrate, pH 6.0 | 0.371 | 1.219 | 0.013 | 0.210 |

Viscosity of the Different IDEC-114 Formulations

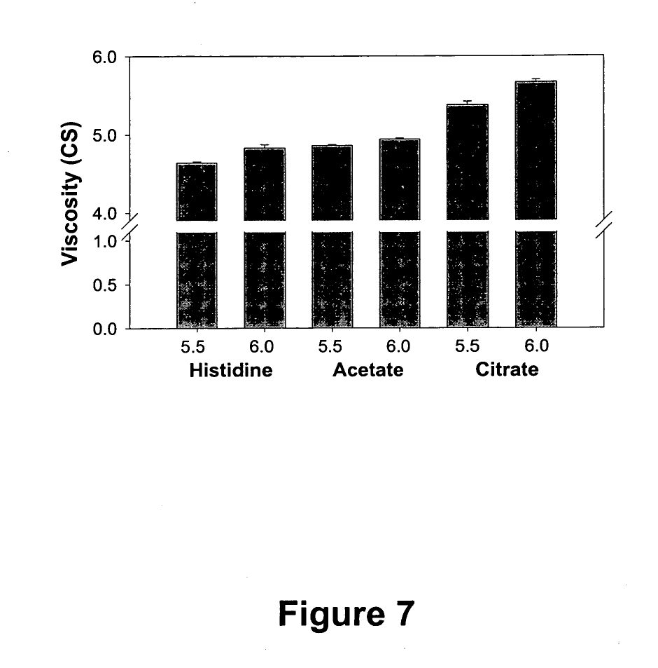

The kinematic viscosities of the samples concentrated to 150 mg/ml were measured by a calibrated size 2 Cross Arm Viscometer (VWR). All the measurements were done at room temperature (23±3° C.) with 3 ml of solution. FIG. 7 is a bar graph that shows the measured kinematic viscosity of the 6 different formulations for IDEC-114. As expected, the citrate-formulated solutions had the highest viscosity, followed by the acetate solutions, and the histidine-buffered solutions had the lowest viscosity. Again, within the same buffer species, pH seemed have an effect on the viscosity, however across the buffer species no specific pH trend could be found.

1. A concentrated antibody composition consisting essentially of an aqueous solution of antibodies and histidine or acetate buffer at a concentration in the range of from about 2 mM to about 48 mM.

2-21. (canceled)

22. A method for producing a concentrated antibody preparation comprising the steps of:
a) providing an initial antibody preparation consisting essentially an aqueous solution of antibodies and histidine or acetate buffer at a concentration in the range of from about 2 mM to about 48 mM; and
b) subjecting the initial antibody preparation to membrane filtration that removes water and buffer but not antibodies from the antibody preparation,
thereby producing an antibody preparation having a higher concentration of antibodies than the initial antibody preparation.

23. The method of claim 22, wherein the concentration of histidine or acetate buffer in the initial antibody preparation is in the range of from about 3 mM to about 48 mM.

24. The method of claim 22, wherein the concentration of histidine or acetate buffer in the initial antibody preparation is in the range of from about 4 mM to about 45 mM.

25. The method of claim 22, wherein the concentration of histidine or acetate buffer in the initial antibody preparation is in the range of from about 5 mM to about 40 mM.

26. The method of claim 22, wherein the concentration of histidine or acetate buffer in the initial antibody preparation is in the range of from 20 mM to 25 mM.

27. The method of claim 22, wherein the pH of the initial antibody preparation is in the range of from about 4.0 to 7.5.

28. The method of claim 22, wherein the pH of the initial antibody preparation is in the range of from 4.5 to 7.0.

29. The method of claim 22, wherein the pH of the initial antibody preparation is in the range of from 5.0 to 6.5.

30. The method of claim 22, wherein the pH of the initial antibody preparation is in the range of from 5.5 to 6.0.

31. The method of claim 22, wherein the antibodies are monoclonal antibodies.

32. The method of claim 31, wherein the antibodies are chimeric antibodies comprising variable regions of a non-human species and human constant regions.

33. The method of claim 32, wherein the antibodies are chimeric antibodies comprising variable regions of an Old World monkey and human constant regions.

34. The method of claim 31, wherein the antibodies are humanized antibodies comprising hypervariable regions of a non-human species, at least one human framework region and human constant regions.

35. The method of claim 22, wherein the antibodies are of one or more of the isotypes selected from IgG, IgM, IgA, IgD, and IgE.

36. The method of claim 35, wherein the antibodies are IgG antibodies.

37. The method of claim 36, wherein the antibodies are IgG₁ or IgG₄ antibodies.

38. The method of claim 22, wherein the concentration of the antibodies in the antibody preparation produced by step b) is at least 50 mg/ml.

39. The method of claim 22, wherein the concentration of the antibodies in the antibody preparation produced by step b) is at least 100 mg/ml.

40. The method of claim 22, wherein the antibodies comprise monoclonal antibodies selected from the group consisting of anti-CD80, anti-gp39, anti-CD4, anti-CD23, and anti-CD20 antibodies.

41. The method of claim 22, wherein the antibodies comprise at least one monoclonal antibody selected from the group consisting the anti-CD80 antibody IDEC-114, the anti-gp39 antibody IDEC-131, the anti-CD4 antibody IDEC 151, the anti-CD23 antibody IDEC-152, and the anti-CD20 antibody rituximab.

42. An improved method for producing a concentrated antibody preparation comprising the steps of:
a) providing an initial antibody preparation consisting essentially of an aqueous solution of antibodies and buffer; and
b) subjecting the initial antibody preparation to membrane filtration that removes water and buffer but not the antibodies from the antibody preparation,
thereby producing an antibody preparation having a higher concentration of antibodies than the initial antibody preparation;
the improvement consisting of using buffer selected from histidine or acetate at a concentration in the range of from about 2 mM to about 48 mM.

43. The improved method of claim 42, wherein the concentration of histidine or acetate buffer in the initial antibody preparation is in the range of from about 3 mM to about 48 mM.

44. The improved method of claim 42, wherein the concentration of histidine or acetate buffer in the initial antibody preparation is in the range of from about 4 mM to about 45 mM.

45. The improved method of claim 42, wherein the concentration of histidine or acetate buffer in the initial antibody preparation is in the range of from about 5 mM to about 40 mM.

46. The improved method of claim 42, wherein the concentration of histidine or acetate buffer in the initial antibody preparation is in the range of from 20 mM to 25 mM.

47. The improved method of claim 42, wherein the pH of the initial antibody preparation is in the range of from about 4.0 to 7.5.

48. The improved method of claim 42, wherein the pH of the initial antibody preparation is in the range of from 4.5 to 7.0.

49. The improved method of claim 42, wherein the pH of the initial antibody preparation is in the range of from 5.0 to 6.5.

50. The improved method of claim 42, wherein the pH of the initial antibody preparation is in the range of from 5.5 to 6.0.

51. The improved method of claim 42, wherein the antibodies are monoclonal antibodies.

52. The improved method of claim 51, wherein the antibodies are chimeric antibodies comprising variable regions of a non-human species and human constant regions.

53. The improved method of claim 52, wherein the antibodies are chimeric antibodies comprising variable regions of an Old World monkey and human constant regions.

54. The improved method of claim 51, wherein the antibodies are humanized antibodies comprising hypervariable regions of a non-human species, at least one human framework region and human constant regions.

55. The improved method of claim 42, wherein the antibodies are of one or more of the isotypes selected from IgG, IgM, IgA, IgD, and IgE.

56. The improved method of claim 55, wherein the antibodies are IgG antibodies.

57. The improved method of claim 56, wherein the antibodies are IgG₁ or IgG₄ antibodies.

58. The improved method of claim 42, wherein the concentration of the antibodies in the antibody preparation produced by step b) is at least 50 mg/ml.

59. The improved method of claim 42, wherein the concentration of the antibodies in the antibody preparation produced by step b) is at least 100 mg/ml.

60. The improved method of claim 42, wherein the antibodies comprise monoclonal antibodies selected from the group consisting of anti-CD80, anti-gp39, anti-CD4, anti-CD23, and anti-CD20 antibodies.

61. The improved method of claim 42, wherein the antibodies comprise at least one monoclonal antibody selected from the group consisting the anti-CD80 antibody IDEC-114, the anti-gp39 antibody IDEC-131, the anti-CD4 antibody IDEC 151, the anti-CD23 antibody IDEC-152, and the anti-CD20 antibody rituximab.

62. A method for producing a pharmaceutical composition comprising antibodies as the active ingredient, comprising the steps of:
a) providing an initial antibody preparation consisting essentially of an aqueous solution of antibodies and histidine or acetate buffer at a concentration in the range of from about 2 mM to about 48 mM; and
b) subjecting the initial antibody preparation to membrane filtration that removes water and buffer but not antibodies from the antibody preparation, thereby producing an antibody preparation having a higher concentration of antibodies than the initial antibody preparation; and
c) combining antibodies of the concentrated antibody preparation of step b) with one or more pharmaceutically acceptable carriers to produce a pharmaceutical composition.

63. The method of claim 62, wherein the concentration of histidine or acetate buffer in the initial antibody preparation is in the range of from about 3 mM to about 48 mM.

64. The method of claim 62, wherein the concentration of histidine or acetate buffer in the initial antibody preparation is in the range of from about 4 mM to about 45 mM.

65. The method of claim 62, wherein the concentration of histidine or acetate buffer in the initial antibody preparation is in the range of from about 5 mM to about 40 mM.

66. The method of claim 62, wherein the concentration of histidine or acetate buffer in the initial antibody preparation is in the range of from 20 mM to 25 mM.

67. The method of claim 62, wherein the pH of the initial antibody preparation is in the range of from about 4.0 to 7.5.

68. The method of claim 62, wherein the pH of the initial antibody preparation is in the range of from 4.5 to 7.0.

69. The method of claim 62, wherein the pH of the initial antibody preparation is in the range of from 5.0 to 6.5.

70. The method of claim 62, wherein the pH of the initial antibody preparation is in the range of from 5.5 to 6.0.

71. The method of claim 62, wherein the antibodies are monoclonal antibodies.

72. The method of claim 71, wherein the antibodies are chimeric antibodies comprising variable regions of a non-human species and human constant regions.

73. The method of claim 72, wherein the antibodies are chimeric antibodies comprising variable regions of an Old World monkey and human constant regions.

74. The method of claim 71, wherein the antibodies are humanized antibodies comprising hypervariable regions of a non-human species, at least one human framework region and human constant regions.

75. The method of claim 62, wherein the antibodies are of one or more of the isotypes selected from IgG, IgM, IgA, IgD, and IgE.

76. The method of claim 75, wherein the antibodies are IgG antibodies.

77. The method of claim 76, wherein the antibodies are IgG₁ or IgG₄ antibodies.

78. The method of claim 62, wherein the concentration of the antibodies in the antibody preparation produced by step b) is at least 50 mg/ml.

79. The method of claim 62, wherein the concentration of the antibodies in the antibody preparation produced by step b) is at least 100 mg/ml.

80. The method of claim 62, wherein the antibodies comprise monoclonal antibodies selected from the group consisting of anti-CD80, anti-gp39, anti-CD4, anti-CD23, and anti-CD20 antibodies.

81. The method of claim 62, wherein the antibodies comprise at least one monoclonal antibody selected from the group consisting the anti-CD80 antibody IDEC-114, the anti-gp39 antibody IDEC-131, the anti-CD4 antibody IDEC 151, the anti-CD23 antibody IDEC-152, and the anti-CD20 antibody rituximab.

82. An improved method of therapy that includes the administration of a pharmaceutical composition comprising an antibody, the improvement comprising administering a pharmaceutical composition that is made by combining
a) an antibody preparation consisting essentially of an aqueous solution containing at least one therapeutically effective dose of an antibody and histidine or acetate buffer at a concentration in the range of from about 2 mM to about 48 mM that has been concentrated by membrane filtration, and
b) one or more pharmaceutically acceptable carriers to produce a pharmaceutical composition.

83-101. (canceled)

102. A kit useful for the treatment of a mammal suffering from or predisposed to a disorder comprising at least one container containing a pharmaceutical composition that is the product of combining:
a) an antibody preparation consisting essentially of an aqueous solution containing at least one therapeutically effective dose of an antibody and histidine or acetate buffer at a concentration in the range of from about 2 mM to about 48 mM that has been concentrated by membrane filtration, and
b) one or more pharmaceutically acceptable carriers;
and further comprises a label or an insert indicating that said pharmaceutical composition may be used to treat said disorder.

103-121. (canceled)
